(19) United States
(12) Patent Application Publication
(10) Pub. No.: US 2011/0287084 A1
(43) Pub. Date: Nov. 24, 2011

(54) METHOD FOR REDUCING BODY FAT AND INCREASING LEAN BODY MASS BY REDUCING STEAROYL-COA DESATURASE 1 ACTIVITY

(76) Inventors: James M. Ntambi, Madison, WI (US); Alan D. Attie, Madison, WI (US); Makoto Miyazaki, Madison, WI (US); Jonathan P. Stoehr, Madison, WI (US)

(21) Appl. No.: 13/096,056

(22) Filed: Apr. 28, 2011

Related U.S. Application Data
(63) Continuation of application No. 12/405,576, filed on Mar. 17, 2009, now abandoned.
Application No. 12/405,576 is a division of application No. 10/094,841, filed on Mar. 8, 2002, now abandoned.
Application No. 10/094,841 is a continuation-in-part of application No. 09/792,468, filed on Feb. 23, 2001, now Pat. No. 6,987,001.
(60) Provisional application No. 60/184,526, filed on Feb. 24, 2000.
(60) Provisional application No. 60/221,697, filed on Jul. 31, 2000.
(60) Provisional application No. 60/225,771, filed on Aug. 17, 2000.

(51) Int. Cl. A61K 31/713; A61P 3/06; A61K 9/127

(52) U.S. Cl. 424/450; 514/44 A

(57) ABSTRACT

It is disclosed here that inhibiting the activity of the enzyme stearoyl-CoA desaturase (SCD1) in an animal causes the animal to have less body fat and greater lean body mass. The lower of SCD1 activity level can be accomplished by inhibiting activity of the enzyme or lowering levels of active enzyme in the subject.

### CROSS-REFERENCE TO RELATED APPLICATIONS

This application is a continuation-in-part application of Ser. No. 09/792,468 filed Feb. 23, 2001.

### STATEMENT REGARDING FEDERALLY SPONSORED RESEARCH OR DEVELOPMENT

To be determined.

### BACKGROUND OF THE INVENTION

Acyl desaturase enzymes catalyze the formation of double bonds in fatty acids derived from either dietary sources or de novo synthesis in the liver. Mammals synthesize four desaturases of differing chain length specificity that catalyze the addition of double bonds at the Δ9, Δ6, Δ5 and Δ4 positions. Stearoyl-CoA desaturases (SCDs) introduce a double bond in the Δ9-position of saturated fatty acids. The preferred substrates are palmitoyl-CoA (16:0) and stearoyl-CoA (18:0), which are converted to palmitoleoyl-CoA (16:1) and oleoyl-CoA (18:1), respectively. The resulting mono-unsaturated fatty acids are substrates for incorporation into triglycerides, phospholipids, and cholesterol esters.

A number of mammalian SCD genes have been cloned. For example, two genes have been cloned from rat (SCD1, SCD2) and four SCD genes have been isolated from mouse (SCD1, 2, 3, and 4). A single SCD gene, SCD1, has been so far been characterized in humans.

While the basic biochemical role of SCD has been known in rats and mice since the 1970's (Jeffcoat R. and James, A T. 1984. Elsevier Science, 4: 85-112; de Antueno, R J. 1993. Lipids 28(4)285-290), it has not, prior to this invention, been directly implicated in controlling body fat and lean body mass in animals.

### BRIEF DESCRIPTION OF THE SEVERAL VIEWS OF THE DRAWINGS

FIG. 1 illustrates the strategy for the generation of SCD1 null mice

FIG. 2 presents graphically the plasma lipoprotein profiles in SCD1 Knock-out and Asebia Male Mice.

FIG. 3 illustrates the VLDL-triglyceride levels in Asebia (SCD1−/−) and SCD1+/− mice. Plasma lipoproteins were separated by fast performance liquid chromatography and the distribution of triglycerides among lipoproteins in the various density fractions of the mice (n=3) were measured. SCD−/− (open circles), SCD1+/− (filled circles). The lipoprotein peaks for VLDL, LDL and HDL are indicated.

FIG. 4 presents data illustrating the ratio of monounsaturated to saturated fatty acid in mouse plasma (the desaturation index) decreases in a manner directly proportional to the level of SCD activity 1. The graph is a comparison of SCD1 knock-out and Asebia mice to their respective controls.

FIG. 5 presents graphical data on the average body weight of SCD1−/− and SCD1+/+ male mice fed with a regular chow diet and a high fat diet.

FIG. 6 illustrates the average weight of various fat pads from SCD1−/− and SCD1+/+ mice fed with a high fat diet.

FIG. 7 illustrates the location of regulatory sequences and binding sites in homologous region of the mouse SCD1 and human SCD1 promoter and 5′-flanking regions. The top scale denotes the position relative to the transcriptional start site. Important promoter sequence elements are indicated.

FIG. 8 is a graphical representation of data showing that female SCD1 knockout mice had significantly higher body mass than controls fed the same diet.

### DETAILED DESCRIPTION OF THE INVENTION

Controlling Body Fat and/or Lean Body Mass

The present invention discloses that body fat and/or lean body mass in a human or non-human animal can be controlled by modulating stearoyl-CoA desaturase-1 (SCD1) activity in the animal. In meat industry, it is desirable to reduce animal body fat and increase lean body mass when the goal is to produce animals with more lean meat. It is also desirable in many situations in the medical and veterinary arts to reduce fat accumulation in adipocytes. For example, in obese human and non-human animals, adipocytes that accumulate excess lipids can become insulin resistant, a characteristic having many adverse effects. The objective here is to reduce body fat and increase lean body mass in both humans and non-human animals. To simplify the language of the disclosure, the terms “animal” and “subject” will be used here to refer both to humans and to non-human animals.

A specific demonstration of controlling body fat and lean body mass by reducing SCD1 activity is described in the examples below. In one example, knocking out SCD1 gene in mice led to lower body fat level and higher lean body mass in these mice in comparison to control mice. This effect was observed when the mice were fed either a regular chow diet or a high fat diet. The high fat diet caused control mice to accumulate more fat in comparison to control mice on the regular chow diet. However, no increase in body fat level was observed in SCD1 knock-out mice fed with the high fat diet in comparison to SCD1 knock-out mice fed with the regular chow diet. While this effect was achieved by genetic manipulation of the mice, genetic alteration of the subject is not required for the effect to occur. What is necessary is for the level of SCD1 activity in the subject be lowered. When SCD1 activity is lowered, the animal or subject will then tend to have lower body fat and more lean body mass as compared to a comparable subject with unchecked SCD1 activity. This can be done through genetic manipulation or through the use of other transient or semi-permanent modulators of SCD1 activity.

The effect described here is effective for any of the various SCD1 genes in various animal species. The SCD1 cloned from different mammalian species all show a high degree of homology, in any event. For example, the human SCD1 protein and the mouse SCD1 protein show about 87% sequence identity at the amino acid level. From the perspective of desaturizing a saturated fatty acid C_{18:0} to C_{18:1} at the Δ9 position, the activity of SCD1 in different animals are conserved. The resulting mono-unsaturated fatty is a substrate for incorporation into triglycerides, the main component of fats. It is expected that reducing the activity of a SCD1 can be used as a method for controlling body fat, lean body mass, or both, in an animal in general. The animals include but are not limited to mammals and avian animals. The mammals include but are not limited to human beings, primates, bovines, canines, porcines, ovines, caprines, felines and rodents.

In the specific example described below, the SCD1 activity was knocked out completely and a reduction in body fat and an increase in lean body mass were observed. It is expected that when SCD1 activity is reduced to a lesser degree, the effects on body fat reduction and lean body mass increase may be attenuated. It is also expected that at a certain level of SCD1 activity reduction, unless body fat reduction and lean body mass increase are equally sensitive to SCD1 activity reduction, one may only observe one effect but not the other. The relative sensitivity of body fat reduction and lean body mass increase to SCD1 activity reduction may be different in different animals. The relationship between body fat reduction/lean body mass increase and different levels SCD1 activity reduction can be readily established by a skilled artisan through conducting routine dose response experiments.

Although under most circumstances, a sufficient reduction in SCD1 activity will result in a reduction in body fat, an increase in lean body mass, or both, when compared to the body fat level and the lean body mass before SCD1 activity reduction, there are circumstances under which the body fat reduction and the lean body mass increase effects of reducing SCD1 activity will not be reflected as above. For example, when an animal is on high fat diet and its body fat level is increasing dramatically, a reduction in SCD1 activity may result in a body fat level lower than what it would have been without SCD 1 activity reduction but higher if compared to the body fat level before SCD1 activity reduction. A similar situation may happen to lean body mass increase. Under these circumstances, the body fat reduction and lean body mass increase effects of reducing SCD 1 activity are measured by comparing the body fat level and the lean body mass, or the rate of body fat increase and lean body mass increase, in animals whose SCD1 activity has been reduced and animals whose SCD 1 activity has not been reduced.

According to the above discussion, the present invention provides that body fat, lean body mass or both in a human or non-human animal can be controlled by reducing SCD1 activity in the animal. By controlling body fat in an animal, we mean at least one of the following three effects: one, reducing body fat to a level lower than that just before SCD1 activity is reduced in the animal; two, maintaining body fat at a level substantially the same as that just before SCD1 activity is reduced in the animal; and three, keeping body fat level lower than what it would have been without reducing SCD1 activity in the animal By controlling lean body mass in an animal, we mean at least one of the following three effects: one, increasing lean body mass to a level higher than that just before SCD1 activity is reduced in the animal; two, maintaining lean body mass at a level substantially the same as that just before SCD1 activity is reduced in the animal; and three, keeping lean body mass level higher than what it would have been without reducing SCD1 activity in the animal. By controlling both body fat and lean body mass in an animal, we mean an effect selected from the three effects for controlling body fat and an effect selected from the three effects for controlling lean body mass.

Any agent that are known to a skilled artisan to reduce SCD1 activity can be used in the present invention. New agents identified to be able to reduce SCD1 activity can also be also be used. Agents can be administered orally, as a food supplement or adjuvant, or by any other effective means which has the effect of reducing SCD1 activity.

While it is envisaged that any suitable mechanism for reducing SCD1 activity can be used, three specific examples of reduction classes are envisioned. One class includes lowering SCD1 protein level. A second class includes the inhibition of SCD1 enzymatic activity. Finally, the third class includes interfering with the proteins essential to the desaturase system, such as cytochrome b₅, NADH (P)-cytochrome b₅ reductase, and terminal cyanide-sensitive desaturase.

Many strategies are available to lower SCD1 protein level. For example, one can increase the degradation rate of the enzyme or inhibit rate of synthesis of the enzyme. The synthesis of the enzyme can be inhibited at transcriptional level or translational level by known genetic techniques. Since SCD1 is regulated by several known transcription factors (e.g. PPAR-γ, SREBP), any agent that affects the activity of such transcription factors can be used to alter the expression of the SCD1 gene at transcriptional level. One group of such agents includes thiazoladine compounds which are known to activate PPAR-γ and inhibit SCD1 transcription. These compounds include Pioglitazone, Ciglitazone, Englitazone, Troglitazone, and BRL49653. Other inhibitory agents may include polyunsaturated fatty acids, such as linoleic acid, arachidonic acid and dodecahexaenoic acid, which also inhibit SCD1 transcription.

One common method that can be used to block the synthesis of a SCD1 protein at translational level is to use an antisense oligonucleotide (DNA or RNA) having a sequence complementary to at least part of the SCD1 mRNA sequence. One of ordinary skill in the art knows how to make and use an antisense oligonucleotide to block the synthesis of a protein (Agarwal, S. (1996) Antisense Therapeutics. Totowa, N.J., Humana Press, Inc.). An example of the antisense method for the present invention is to use 20-25 mer antisense oligonucleotides directed against 5′ end of the SCD1 message with phosphorothioate derivatives on the last three base pairs on the 3′ end and the 5′ end to enhance the half life and stability of the oligonucleotides. A useful strategy is to design several oligonucleotides with a sequence that extends 2-5 basepairs beyond the 5′ start site of transcription.

An antisense oligonucleotide used for controlling body fat level and/or lean body mass can be administered intravenously into an animal. A carrier for an antisense oligonucleotide can be used. An example of a suitable carrier is cationic liposomes. For example, an oligonucleotide can be mixed with cationic liposomes prepared by mixing 1-alpha dioleylphatidylcelthanolamine with dimethldioctadecylammonium bromide in a ratio of 5:2 in 1 ml of chloroform. The solvent will be evaporated and the lipids resuspended by sonication in 10 ml of saline.

Another way to use an antisense oligonucleotide is to engineer it into a vector so that the vector can produce an antisense cRNA that blocks the translation of the mRNAs encoding for SCD1.

For effectively inhibiting the enzymatic activity of the SCD1 protein, it is envisaged that any agent capable of disrupting the activity of the SCD1 protein could be utilized. For example, certain conjugated linoleic acid isomers are effective inhibitors of SCD1 activity. Specifically, Cis-12, trans-10 conjugated linoleic acid is known to effectively inhibit SCD enzyme activity and reduce the abundance of SCD1 mRNA while Cis-9, trans-11 conjugated linoleic acid does not. Cyclopropenoid fatty acids, such as those found in stercula and cotton seeds, are also known to inhibit SCD activity. For example, sterculic acid (8-(2-octyl-cyclopropenyl)octanoic acid) and Malvalic acid (7-(2-octyl-cyclopropenyl)heptanoic acid) are C18 and C16 derivatives of sterculoyl- and malvaloyl-fatty acids, respectively, having cyclopropene rings at their Δ9 position. These agents inhibit SCD activity by inhibiting Δ9 desaturation. Other agents include thia-fatty acids, such as 9-thiastearic acid (also called 8-nonylthiooctanoic acid) and other fatty acids with a sulfoxy moiety.

The known modulators of delta-9 desaturase activity are either not known to be useful for controlling body fat and lean body mass as claimed in this invention, or else they are otherwise unsatisfactory therapeutic agents. The thia-fatty acids, conjugated linoleic acids and cyclopropene fatty acids (malvalic acid and sterculic acid) are neither useful at reasonable physiological doses, nor are they specific inhibitors of SCD1 biological activity, rather they demonstrate cross inhibition of other desaturases, in particular the delta-5 and delta-6 desaturases by the cyclopropene fatty acids. These compounds may be useful for establishing control or test modulators of the screening assays of the invention, but are not subject to the claims of this invention. Preferred SCD1 inhibitors of the invention have no significant or substantial impact on unrelated classes of proteins. In some cases, assays specific for the other proteins, such as delta-5 and delta-6 activity, will also have to be tested to ensure that the identified compounds of the invention do not demonstrate significant or substantial cross inhibition.

The known non-specific inhibitors of SCD1 can be useful in rational design of a therapeutic agent suitable for inhibition of SCD1. All three inhibitors have various substitutions between carbons #9 and #10; additionally they require conjugation to Co-A to be effective; and are probably situated in a relatively hydrophobic active site. This information combined with the known X-ray co-ordinates for the active site for plant (soluble) SCD can assist the “in silico” process of rational drug design for therapeutically acceptable inhibitors specific for SCD1.

Besides the SCD1 enzyme inhibitors described above, a SCD1 monoclonal or polyclonal antibody, or an SCD1-binding fragment thereof, can also be used as enzyme inhibitors for the purpose of this invention. In one embodiment, the antibody is isolated, i.e., an antibody free of any other antibodies. Generally, it has been shown that an antibody can block the function of a target protein when administered into the body of an animal Dahly, A. J., FASEB J. 14:A133, 2000; Dahly, A. J., J. Am. Soc. Nephrology 11:332A, 2000. Thus, a SCD1 antibody can be used to control body fat level and lean body mass. For example, about 0.01 mg to about 100 mg, preferably about 0.1 mg to about 10 mg, and most preferably about 0.2 mg to about 1.0 mg of humanized SCD1 antibodies can be administered into an animal The half life of these antibodies in a human being can be as long as 2-3 weeks. For the SCD1s whose DNA and protein amino acid sequences are published and available, one of ordinary skill in the art knows how to make monoclonal or polyclonal antibodies against them (Harlow, et al. 1988. Antibodies: A Laboratory Manual; Cold Spring Harbor, N.Y., Cold Spring Harbor Laboratory).

### Screening Assays

Since the present invention is based on reducing SCD1 activity levels, screening assays employing the SCD1 gene and/or protein for use in identifying agents for use in controlling body fat and lean body mass in an animal are useful in performing this process.

1. “SCD1 Biological Activity”

“SCD1 biological activity” as used herein, especially relating to screening assays, is interpreted broadly and contemplates all directly or indirectly measurable and identifiable biological activities of the SCD1 gene and protein. Relating to the purified SCD1 protein, SCD1 biological activity includes, but is not limited to, all those biological processes, interactions, binding behavior, binding-activity relationships, pKa, pD, enzyme kinetics, stability, and functional assessments of the protein. Relating to SCD1 biological activity in cell fractions, reconstituted cell fractions or whole cells, these activities include, but are not limited the rate at which the SCD introduces a cis-double bond in its substrates palmitoyl-CoA (16:0) and stearoyl-CoA (18:0), which are converted to palmitoleoyl-CoA (16:1) and oleoyl-CoA (18:1), respectively, and all measurable consequences of this effect, such as triglyceride, cholesterol, or other lipid synthesis, membrane composition and behavior, cell growth, development or behavior and other direct or indirect effects of SCD1 activity. Relating to SCD1 genes and transcription, SCD1 biological activity includes the rate, scale or scope of transcription of genomic DNA to generate RNA; the effect of regulatory proteins on such transcription, the effect of modulators of such regulatory proteins on such transcription; plus the stability and behavior of mRNA transcripts, post-transcription processing, mRNA amounts and turnover, and all measurements of translation of the mRNA into polypeptide sequences. Relating to SCD1 biological activity in organisms, this includes but is not limited biological activities which are identified by their absence or deficiency in disease processes or disorders caused by aberrant SCD1 biological activity in those organisms. Broadly speaking, SCD1 biological activity can be determined by all these and other means for analyzing biological properties of proteins and genes that are known in the art.

2. Design and Development of SCD Screening Assays

The present disclosure facilitates the development of screening assays that may be cell based, cell extract (i.e. microsomal assays), cell free (i.e. transcriptional) assays, and assays of substantially purified protein activity. Such assays are typically radioactivity or fluorescence based (i.e. fluorescence polarization or fluorescence resonance energy transfer or FRET), or they may measure cell behavior (viability, growth, activity, shape, membrane fluidity, temperature sensitivity etc). Alternatively, screening may employ multicellular organisms, including genetically modified organisms such as knock-out or knock-in mice, or naturally occurring genetic variants. Screening assays may be manual or low throughput assays, or they may be high throughput screens which are mechanically/robotically enhanced.

The aforementioned processes afford the basis for screening processes, including high throughput screening processes, for determining the efficacy of potential agents for controlling body fat and lean body mass.

The assays disclosed herein essentially require the measurement, directly or indirectly, of an SCD1 biological activity. Those skilled in the art can develop such assays based on well known models, and many potential assays exist. For measuring whole cell activity of SCD1 directly, methods that can be used to quantitatively measure SCD activity include for example, measuring thin layer chromatographs of SCD reaction products over time. This method and other methods suitable for measuring SCD activity are well known (Henderson Henderson “R J, et al. 1992. Lipid Analysis: A Practical Approach. Hamilton S. Eds. New York and Tokyo, Oxford University Press. pp 65-111.). Gas chromatography is also useful to distinguish monounsaturates from saturates, for example oleate (18:1) and stearate (18:0) can be distinguished using this method. A description of this method is in the examples below. These techniques can be used to determine if a test compound has influenced the biological activity of SCD1, or the rate at which the SCD introduces a cis-double bond in its substrate palmitate (16:0) or stearate (18:0) to produce palmitolyeoyl-CoA (16:1) or oleyoyl-CoA (18:1), respectively.

In one embodiment of an SCD1 activity assay, the assay employs a microsomal assay having a measurable SCD1 biological activity. A suitable assay may be taken by modifying and scaling up the rat liver microsomal assay essentially as described by Shimomura et al. (Shimomura, I., Shimano, H., Korn, B. S., Bashmakov, Y., and Horton, J. D. (1998). Tissues are homogenized in 10 vol. of buffer A (0.1M potassium buffer, pH 7.4). The microsomal membrane fractions (100,000×g pellet) are isolated by sequential centrifugation. Reactions are performed at 37° C. for 5min with 100 μg of protein homogenate and 60 μM of [1-14C]-stearoyl-CoA (60,000 dpm), 2mM of NADH, 0.1M of Tris/HCI buffer (pH 7.2). After the reaction, fatty acids are extracted and then methylated with 10% acetic chloride/methanol. Saturated fatty acid and monounsaturated fatty acid methyl esters are separated by 10% AgNO₃-impregnated TLC using hexane/diethyl ether (9:1) as developing solution. The plates are sprayed with 0.2% 2′,7′-dichlorofluorescein in 95% ethanol and the lipids are identified under UV light. The fractions are scraped off the plate, and the radioactivity is measured using a liquid scintillation counter.

Specific embodiments of such SCD1 biological activity assay take advantage of the fact that the SCD reaction produces, in addition to the monounsaturated fatty acyl-CoA product, H₂O. If ³H is introduced into the C-9 and C-10 positions of the fatty-acyl-CoA substrate, then some of the radioactive protons from this reaction will end up in water. Thus, the measurement of the activity would involve the measurement of radioactive water. In order to separate the labeled water from the stearate, investigators may employ substrates such as charcoal, hydrophobic beads, or just plain old-fashioned solvents in acid pH.

In another embodiment, screening assays measure SCD1 biological activity indirectly. Standard high-throughput screening assays center on ligand-receptor assays. These may be fluorescence based or luminescence based or radiolabel detection. Enzyme immunoassays can be run on a wide variety of formats for identifying compounds that interact with SCD1 proteins. These assays may employ prompt fluorescence or time-resolved fluorescence immunoassays which are well known. P³² labeled ATP, is typically used for protein kinase assays. Phosphorylated products may be separated for counting by a variety of methods. Scintillation proximity assay technology is an enhanced method of radiolabel assay. All these types of assays are particularly appropriate for assays of compounds that interact with purified or semi-purified SCD1 protein.

In yet another embodiment, the assay makes use of ³H-stearoyl CoA (with the ³H on the 9 and 10 carbon atoms), the substrate for SCD1. Desaturation by SCD1, produces oleoyl CoA and ³H -water molecules. The reaction is run at room temperature, quenched with acid and then activated charcoal is used to separate un reacted substrate from the radioactive water product. The charcoal is sedimented and amount of radioactivity in the supernatant is determined by liquid scintillation counting. This assay is specific for SCD1-dependent desaturation as judged by the difference seen when comparing the activity in wild type and SCD1-knockout tissues. Further, the method is easily adapted to high throughput as it is cell-free, conducted at room temperature and is relatively brief (1 hour reaction time period versus previous period of 2 days.

While the instant disclosure sets forth an effective working embodiment of the invention, those skilled in the art are able to optimize the assay in a variety of ways, all of which are encompassed by the invention. For example, charcoal is very efficient (>98%) at removing the unused portion of the stearoyl-CoA but has the disadvantage of being messy and under some conditions difficult to pipette. It may not be necessary to use charcoal if the stearoyl-CoA complex sufficiently aggregates when acidified and spun under moderate g-force. This can be tested by measuring the signal/noise ratio with and without charcoal following a desaturation reaction. There are also other reagents that would efficiently sediment stearoyl-CoA to separate it from the ³H -water product.

The following assays are also suitable for measuring SCD1 biological activity in the presence of potential agents. These assays are also valuable as secondary screens to further select SCD1 specific inhibitors from a library of potential therapeutic agents.

Cellular based desaturation assays can be used to track SCD1 activity levels. By tracking the conversion of stearate to oleate in cells (3T3L1 adipocytes are known to have high SCD1 expression and readily take up stearate when complexed to BSA) one can evaluate compounds found to be inhibitory in the primary screen for additional qualities or characteristics such as whether they are cell permeable, lethal to cells, and/or competent to inhibit SCD1 activity in cells. This cellular based assay may employ a recombinant cell line containing a delta-9 desaturase. The recombinant gene is optionally under control of an inducible promoter and the cell line preferably over-expresses SCD1 protein.

Other assays for tracking other SCD isoforms can be developed. For example, rat and mouse SCD2 is expressed in brain. A microsome preparation can be made from the brain as previously done for SCD1 from liver. The object may be to find compounds that would be specific to SCD1. This screen would compare the inhibitory effect of the compound for SCD1 versus SCD2.

Although unlikely, it is possible that a compound “hit” in the SCD1 assay may result from stimulation of an enzyme present in the microsome preparation that competitively utilizes stearoyl-CoA at the expense of that available for SCD1-dependent desaturation. This would mistakenly be interpreted as SCD1 inhibition. One possibility to examine this problem would be incorporation into phospholipids of the unsaturated lipid (stearate). By determining effects of the compounds on stimulation of stearate incorporation into lipids researchers are able to evaluate this possibility.

Cell based assays may be preferred, for they leave the SCD1 gene in its native format. Particularly promising for SCD1 analysis in these types of assays are fluorescence polarization assays. The extent to which light remains polarized depends on the degree to which the tag has rotated in the time interval between excitation and emission. Since the measurement is sensitive to the tumbling rate of molecules, it can be used to measure changes in membrane fluidity characteristics that are induced by SCD1 activity—namely the delta-9 desaturation activity of the cell. An alternate assay for SCD1 involves a FRET assay. FRET assays measure fluorescence resonance energy transfer which occurs between a fluorescent molecule donor and an acceptor, or quencher. Such an assay may be suitable to measure changes in membrane fluidity or temperature sensitivity characteristics induced by SCD1 biological activity.

The screening assays of the invention may be conducted using high throughput robotic systems. In the future, preferred assays may include chip devices developed by, among others, Caliper, Inc., ACLARA BioSciences, Cellomics, Inc., Aurora Biosciences Inc., and others.

In other embodiments of an SCD1 assay, SCD1 biological activity can also be measured through a cholesterol efflux assay that measures the ability of cells to transfer cholesterol to an extracellular acceptor molecule and is dependent on ABCA1 function. A standard cholesterol efflux assay is set out in Marcil et al., Arterioscler. Thromb. Vasco Bioi. 19:159-169, 1999, incorporated by reference herein for all purposes.

Preferred assays are readily adapted to the format used for drug screening, which may consist of a multi-well (e.g., 96-well, 384 well or 1,536 well or greater) format. Modification of the assay to optimize it for drug screening would include scaling down and streamlining the procedure, modifying the labeling method, altering the incubation time, and changing the method of calculating SCD1 biological activity and so on. In all these cases, the SCD1 biological activity assay remains conceptually the same, though experimental modifications may be made.

Another preferred cell based assay is a cell viability assay for the isolation of SCD1 inhibitors. Overexpression of SCD decreases cell viability. This phenotype can be exploited to identify inhibitory compounds. This cytotoxicity may be due to alteration of the fatty acid composition of the plasma membrane. In a preferred embodiment, the human SCD1 cDNA would be placed under the control of an inducible promoter, such as the Tet-On Tet-Off inducible gene expression system (Clontech). This system involves making a double stable cell line. The first transfection introduces a regulator plasmid and the second would introduce the inducible SCD expression construct. The chromosomal integration of both constructs into the host genome would be favored by placing the transfected cells under selective pressure in the presence of the appropriate antibiotic. Once the double stable cell line was established, SCD1 expression would be induced using the tetracycline or a tetracycline derivative (e.g., Doxycycline). Once SCD1 expression had been induced, the cells would be exposed to a library of chemical compounds for high throughput screen of potential inhibitors. After a defined time period, cell viability would then be measured by means of a fluorescent dye or other approach (e.g., turbidity of the tissue culture media). Those cells exposed to compounds that act to inhibit SCD1 activity would show increased viability, above background survival. Thus, such an assay would be a positive selection for inhibitors of SCD1 activity based on inducible SCD1 expression and measurement of cell viability.

An alternative approach is to interfere with the desaturase system. The desaturase system has three major proteins: cytochrome b₅, NADH (P)-cytochrome b₅ reductase, and terminal cyanide-sensitive desaturase. Terminal cyanide-sensitive desaturase is the product of the SCD gene. SCD activity depends upon the formation of a stable complex between the three aforementioned components. Thus, any agent that interferes with the formation of this complex or any agent that interferes with the proper function of any of the three components of the complex would effectively inhibit SCD activity.

Another type of modulator of SCD1 activity involves a 33 amino acid destabilization domain located at the amino terminal end of the pre-SCD1 protein (Mziaut et al., PNAS 2000, 97: p 8883-8888). It is possible that this domain may be cleaved from the SCD1 protein by an as yet unknown protease. This putative proteolytic activity would therefore act to increase the stability and half-life of SCD1 Inhibition of the putative protease, on the other hand, would cause a decrease in the stability and half life of SCD1. Compounds which block or modulate removal of the destabilization domain therefore will lead to reductions in SCD1 protein levels in a cell. Therefore, in certain embodiments of the invention, a screening assay will employ a measure of protease activity to identify modulators of SCD1 protease activity. The first step is to identify the specific protease which is responsible for cleavage of SCD1. This protease can then be integrated into a screening assay. Classical protease assays often rely on splicing a protease cleavage site (i.e., a peptide containing the cleavable sequence pertaining to the protease in question) to a protein, which is deactivated upon cleavage. A tetracycline efflux protein may be used for this purpose. A chimera containing the inserted sequence is expressed in E. coli. When the protein is cleaved, tetracycline resistance is lost to the bacterium. In vitro assays have been developed in which a peptide containing an appropriate cleavage site is immobilized at one end on a solid phase. The other end is labeled with a radioisotope, fluorophore, or other tag. Enzyme-mediated loss of signal from the solid phase parallels protease activity. These techniques can be used both to identify the protease responsible for generating the mature SCD1 protein, and also for identifying modulators of this protease for use in decreasing SCD1 levels in a cell.

An SCD1 activity assay may also be carried out as a cell free assay employing a cellular fractional, such as a microsomal fraction, obtained by conventional methods of differential cellular fractionation, most commonly by ultracentrifugation methods.

These results suggest that inhibitors of SCD1 biological activity, such as hSCD1, in a human, may have the beneficial effect of reducing body fat and/or increasing lean body mass. In these human data results, SCD biological activity was measured via the surrogate marker of the ratio of 18:1 to 18:0 fatty acids in the total plasma lipid fraction. This marker indirectly measures SCD1 biological activity.

3. SCD1 Recombinant Cell Lines

In certain embodiments, screening protocols to develop agents to practice the present invention might contemplate use of a SCD1 gene or protein in a recombinant cell line. SCD1 recombinant cell lines may be generated using techniques known in the art, and those more specifically set out below.

The present invention also relates to vectors which contain polynucleotides of the present invention, and host cells which are genetically engineered with vectors of the invention, especially where such cells result in a cell line that can be used for assay of SCD1 activity, and production of SCD1 polypeptides by recombinant techniques.

Host cells are preferably eukaryotic cells, preferably insect cells of Spodoptera species, most especially SF9 cells. Host cells are genetically engineered (transduced or transformed or transfected) with the vectors, (especially baculovirus) of this invention which may be, for example, a cloning vector or an expression vector. Such vectors can include plasmids, viruses and the like. The engineered host cells are cultured in conventional nutrient media modified as appropriate for activating promoters, selecting transformants or amplifying the genes of the present invention. The culture conditions, such as temperature, pH and the like, are those previously used with the host cell selected for expression, and will be apparent to a skilled artisan.

The polynucleotides of the present invention may be employed for producing polypeptides by recombinant techniques. Thus, for example, the polynucleotide may be included in anyone of a variety of expression vectors for expressing a polypeptide. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector may be used as long as it is replicable and viable in the host.

The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art.

The DNA sequence in the expression vector is operatively linked to an appropriate expression control sequence(s) (promoter) to direct mRNA synthesis. As representative examples of such promoters, there may be mentioned: LTR or SV40 promoter, the E. coli lac or trp, the phage lambda P_{L} promoter and other promoters known to control expression of genes in prokaryotic or eukaryotic cells or their viruses. The expression vector also contains a ribosome binding site for translation initiation and a transcription terminator. The vector may also include appropriate sequences for amplifying expression.

In addition, the expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

The vector containing the appropriate DNA sequence as hereinabove described, as well as an appropriate promoter or control sequence, may be employed to transform an appropriate host to permit the host to express the protein. Such transformation will be permanent and thus give rise to a cell line that can be used for further testing.

As representative examples of appropriate hosts, there may be mentioned Spodoptera Sf9 (and other insect expression systems) and animal cells such as CHO, COS or Bowes melanoma; adenoviruses; plant cells, and even bacterial cells, etc, all of which are capable of expressing the polynucleotides disclosed herein. The selection of an appropriate host is deemed to be within the knowledge of those skilled in the art based on the teachings herein. For use in the assay methods disclosed herein, mammalian cells are preferred.

More particularly, the present invention also includes recombinant constructs comprising one or more of the sequences as broadly described above. The constructs comprise a vector, such as a plasmid or viral vector, especially where the Baculovirus/SF9 vector/expression system is used, into which a sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available. The following vectors are provided by way of example; Bacterial: pQE70, pQE60, pQE-9 (Qiagen), pBS, pD10, phagescript, psiX174, pBluescript SK, pBSKS, pNH8A, pNH16a, pNH18A, pNH46A (Stratagene); pTRC99a, pKK223-3, pKK233-3, pDR540, pRIT5 (Pharmacia); Eukaryotic: pWLNEO, pSV2CAT, pOG44, pXT1, pSG (Stratagene) pSVK3, pBPV, pMSG, pSVL (Pharmacia). However, any other plasmid or vector may be used as long as they are replicable and viable in the host.

Promoter regions can be selected from any desired gene using CAT (chloramphenicol transferase) vectors or other vectors with selectable markers. Two appropriate vectors are pKK232-8 and pCM7. Particular named bacterial promoters include lacl, lacZ, T3, T7, gpt, lambda P_{R}, P_{L} and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

In a further embodiment, the present invention relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (Davis, L., Dibner, M., Battey, I., Basic Methods in Molecular Biology, (1986)). A preferred embodiment utilizes expression from insect cells, especially SF9 cells from Spodoptera frugiperda.

The constructs in host cells can be used in a conventional manner to produce the gene product encoded by the recombinant sequence. Alternatively, the polypeptides of the invention can be synthetically produced by conventional peptide synthesizers.

Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., Molecular Cloning: A laboratory Manual, Second Edition, Cold Spring Harbor, N.Y., (1989), Wu et al., Methods in Gene Biotechnology (CRC Press, New York, N.Y., 1997), Recombinant Gene Expression Protocols, In Methods in Molecular Biology, Vol. 62, (Tuan, ed., Humana Press, Totowa, NJ, 1997), and Current Protocols in Molecular Biology, (Ausabel et al., Eds.,), John Wiley & Sons, NY (1994-1999), the disclosures of which are hereby incorporated by reference in their entirety.

Transcription of the DNA encoding the polypeptides of the present invention by eukaryotic cells, especially mammalian cells, most especially human cells, is increased by inserting an enhancer sequence into the vector. Enhancers are cis-acting elements of DNA, usually about from 10 to 300 bp that act on a promoter to increase its transcription. Examples include the SV40 enhancer on the late side of the replication origin by 100 to 270, a cytomegalovirus early promoter enhancer, the polyoma enhancer on the late side of the replication origin, and adenovirus enhancers.

Generally, recombinant expression vectors will include origins of replication and selectable markers permitting transformation of the host cell, e.g., the ampicillin resistance gene of E. coli and S. cerevisiae Trp1 gene, and a promoter derived from a highly-expressed gene to direct transcription of a downstream structural sequence. Such promoters can be derived from operons encoding glycolytic enzymes such as 3-phosphoglycerate kinase (PGK), α-factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal or C-terminal identification peptide imparting desired characteristics, e.g., stabilization or simplified purification of expressed recombinant product.

Use of a Baculovirus-based expression system is a preferred and convenient method of forming the recombinants disclosed herein. Baculoviruses represent a large family of DNA viruses that infect mostly insects. The prototype is the nuclear polyhedrosis virus (AcMNPV) from Autographa californica, which infects a number of lepidopteran species. One advantage of the baculovirus system is that recombinant baculoviruses can be produced in vivo. Following co-transfection with transfer plasmid, most progeny tend to be wild type and a good deal of the subsequent processing involves screening. To help identify plaques, special systems are available that utilize deletion mutants. By way of non-limiting example, a recombinant AcMNPV derivative (called BacPAK6) has been reported in the literature that includes target sites for the restriction nuclease Bsu361 upstream of the polyhedrin gene (and within ORF 1629) that encodes a capsid gene (essential for virus viability). Bsf361 does not cut elsewhere in the genome and digestion of the BacPAK6 deletes a portion of the ORF 1629, thereby rendering the virus non-viable. Thus, with a protocol involving a system like Bsu361-cut BacPAK6 DNA most of the progeny are non-viable so that the only progeny obtained after co-transfection of transfer plasmid and digested BacPAK6 is the recombinant because the transfer plasmid, containing the exogenous DNA, is inserted at the Bsu361 site thereby rendering the recombinants resistant to the enzyme (see Kitts and Possee, A method for producing baculovirus expression vectors at high frequency, BioTechniques, 14,810-817 (1993)). For general procedures, see King and Possee, The Baculovirus Expression System: A Laboratory Guide, Chapman and Hall, New York (1992) and Recombinant Gene Expression Protocols, in Methods in Molecular Biology, Vol. 62, (Tuan, ed., Humana Press, Totowa, N.J., 1997), at Chapter 19, pp. 235-246.

Alternatively, the screening assay may employ a vector construct comprising a SCD1 promoter sequence operably linked to a reporter gene. Such a vector can be used to study the effect of potential transcription regulatory proteins, and the effect of compounds that inhibit the effect of those regulatory proteins, on the transcription of SCD1.

Factors that may modulate gene expression include transcription factors such as, but not limited to, retinoid X receptors (RXRs), peroxisomal proliferation-activated receptor (PPAR) transcription factors, the steroid response element binding proteins (SREBP-1 and SREBP-2), REV-ERBα, ADD-1, EBPα, CREB binding protein, P300, HNF 4, RAR, LXR, and RORα, NF-Y, C/EBPalpha, PUFA-RE and related proteins and transcription regulators. Screening assays designed to assess the capacity of test compounds to inhibit the ability of these transcription factors to transcribe SCD1 are also contemplated by this invention.

In accordance with the foregoing, following identification of chemical agents having the desired inhibiting activity of SCD1, the present invention also relates to a process for treating an animal, especially a human, who is obese involving inhibiting SCD1 activity in said animal In a preferred embodiment, said inhibition of SCD1 activity is not accompanied by substantial inhibition of activity of delta-5 desaturase, delta-6 desaturase or fatty acid synthetase. In a specific embodiment, the present invention relates to a process for controlling body fat and/or lean body mass comprising administering to said animal an effective amount of an agent whose activity was first identified by the process of the invention.

In accordance with the foregoing, the present invention also relates to an inhibitor of SCD1 activity and which is useful for controlling body fat and/or lean body mass wherein said activity was first identified by its ability to inhibit SCD1 activity, especially where such inhibition was first detected using a process as disclosed herein according to the present invention. In a preferred embodiment thereof, such inhibiting agent does not substantially inhibit fatty acid synthetase, delta-5 desaturase or delta-6 desaturase.

In accordance with the foregoing, the present invention further relates to a process for controlling body fat and/or lean body weight in an animal, comprising administering to said animal an effective amount of an agent for which such body fat and/or lean body mass controlling activity was identified by a process as disclosed herein according to the invention.

In a preferred embodiments of such process, the inhibiting agent does not substantially inhibit fatty acid synthetase, delta-5 desaturase or delta-6 desaturase.

4. Test Compounds/Inhibitors/Library Sources

In accordance with the foregoing, the present invention also relates to agents, regardless of molecular size or weight, effective in controlling body fat and/or lean boy mass, and/or diagnosing and/or preventing obesity, preferably where such agents have the ability to inhibit the activity and/or expression of the SCD1, and most preferably where said agents have been determined to have such activity through at least one of the screening assays disclosed according to the present invention.

Test compounds are generally compiled into libraries of such compounds, and a key object of the screening assays of the invention is to select which compounds are relevant from libraries having hundreds of thousands, or millions of compounds.

Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

Thus, in one aspect the present invention relates to agents capable of inhibiting the activity and/or expression of stearoyl-CoA desaturase 1 (SCD1), especially where said inhibiting ability was first determined using an assay of comprising SCD1 or a gene encoding SCD1, or an assay which measures SCD1 activity. As used herein the term “capable of inhibiting” refers to the characteristic of such an agent whereby said agent has the effect of inhibiting the overall biological activity of SCD1, either by decreasing said activity, under suitable conditions of temperature, pressure, pH and the like so as to facilitate such inhibition to a point where it can be detected either qualitatively or quantitatively and wherein such inhibition may occur in either an in vitro or in vivo environment. In addition, while the term “inhibition” is used herein to mean a decrease in activity, the term “activity” is not to be limited to specific enzymatic activity alone (for example, as measured in units per milligram or some other suitable unit of specific activity) but includes other direct and indirect effects of the protein, including decreases in enzyme activity due not to changes in specific enzyme activity but due to changes of expression of polynucleotides encoding and expressing said SCD1 enzyme. Human SCD1 activity may also be influenced by agents which bind specifically to substrates of hSCD1. Thus, the term “inhibition” as used herein means a decrease in SCD1 activity regardless of the molecular genetic level of said inhibition, be it an effect on the enzyme per se or an effect on the genes encoding the enzyme or on the RNA, especially mRNA, involved in expression of the genes encoding said enzyme. Thus, modulation by such agents can occur at the level of DNA, RNA or enzyme protein and can be determined either in vivo or ex vivo.

In specific embodiments thereof, said assay is any of the assays disclosed herein according to the invention. In addition, the agent(s) contemplated by the present disclosure includes agents of any size or chemical character, either large or small molecules, including proteins, such as antibodies, nucleic acids, either RNA or DNA, and small chemical structures, such as small organic molecules.

5. Combinatorial and Medicinal Chemistry

Typically, a screening assay, such as a high throughput screening assay, will identify several or even many compounds which modulate the activity of the assay protein. The compound identified by the screening assay may be further modified before it is used in animals as the therapeutic agent. Typically, combinatorial chemistry is performed on the inhibitor, to identify possible variants that have improved absorption, biodistribution, metabolism and/or excretion, or other important aspects. The essential invariant is that the improved compounds share a particular active group or groups which are necessary for the desired inhibition of the target protein. Many combinatorial chemistry and medicinal chemistry techniques are well known in the art. Each one adds or deletes one or more constituent moieties of the compound to generate a modified analog, which analog is again assayed to identify compounds of the invention. Thus, as used in this invention, compounds identified using an SCD1 screening assay of the invention include actual compounds so identified, and any analogs or combinatorial modifications made to a compound which is so identified which are useful for controlling body ft and/or lean body mass.

6. Pharmaceutical Preparations and Dosages

In another aspect the present invention is directed to compositions comprising the polynucleotides, polypeptides or other chemical agents, including therapeutic, prophylactic or diagnostic agents, such as small organic molecules, disclosed herein according to the present invention wherein said polynucleotides, polypeptides or other agents are suspended in a pharmacologically acceptable carrier, which carrier includes any pharmacologically acceptable diluent or excipient. Pharmaceutically acceptable carriers include, but are not limited to, liquids such as water, saline, glycerol and ethanol, and the like, including carriers useful in forming sprays for nasal and other respiratory tract delivery or for delivery to the ophthalmic system. A thorough discussion of pharmaceutically acceptable carriers, diluents, and other excipients is presented in REMINGTON'S PHARMACEUTICAL SCIENCES (Mack Pub. Co., N.J, current edition).

The inhibitors utilized above may be delivered to a subject using any of the commonly used delivery systems known in the art, as appropriate for the inhibitor chosen. The preferred delivery systems include intravenous injection or oral delivery, depending on the ability of the selected inhibitor to be adsorbed in the digestive tract. Any other delivery system appropriate for delivery of small molecules, such as skin patches, may also be used as appropriate.

In another aspect the present invention further relates to a process for preventing or treating obesity or condition in a patient afflicted therewith comprising administering to said patient a therapeutically or prophylactically effective amount of a composition as disclosed herein.

7. Diagnosis and Pharmacogenomics

In an additional aspect, the present invention also relates to a process for diagnosing a disease or condition in an animal, such as a human being, suspected of being afflicted therewith, or at risk of becoming afflicted therewith, comprising obtaining a tissue sample from said animal and determining the level of activity of SCD1 in the cells of said tissue sample and comparing said activity to that of an equal amount of the corresponding tissue from an animal not suspected of being afflicted with, or at risk of becoming afflicted with, said disease or condition. In specific embodiments thereof, said disease or condition includes, but is not limited to, obesity and low level of lean body mass.

In an additional aspect, this invention teaches that SCD1 has pharmacogenomic significance. Variants of SCD1 including SNPs (single nucleotide polymorphisms), cSNPs (SNPs in a cDNA coding region), polymorphisms and the like may have dramatic consequences on a subject's response to administration of a prophylactic or therapeutic agent. Certain variants may be more or less responsive to certain agents. In another aspect, any or all therapeutic agents may have greater or lesser deleterious side-effects depending on the SCD1 variant present in the subject.

In a pharmacogenomic application of this invention, an assay is provided for identifying cSNPs (coding region small nucleotide polymorph isms) in SCD1 of an individual which are correlated with human disease processes or response to medication. Researchers have identified two putative cSNPs of hSCD1 to date: in exon 1, a C/A SNP at nt 259, corresponding to a D/E amino acid change at position 8; and in exon 5, a C/A cSNP at nt 905, corresponding to a L/M amino acid change at position 224. (Sequence numbering according to GenBank Accession: AF097514). It is anticipated that these putative cSNPs may be correlated with human disease processes or response to medication of individuals who contain those cSNPs versus a control population. Those skilled in the art are able to determine which disease processes and which responses to medication are so correlated.

In carrying out the procedures of the present invention it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

In applying the disclosure, it should be kept clearly in mind that other and different embodiments of the methods disclosed according to the present invention will no doubt suggest themselves to those of skill in the relevant art.

### EXAMPLE 1

### Disruption of Stearoyl-CoA Desaturasel Gene in Mice

This example describes the generation of a SCD1 null (SCD1−/−) mice. Certain lipids and fatty acids in the SCD1 null (knock-out) mice were also analyzed.

Targeted Disruption of the SCD1 Gene

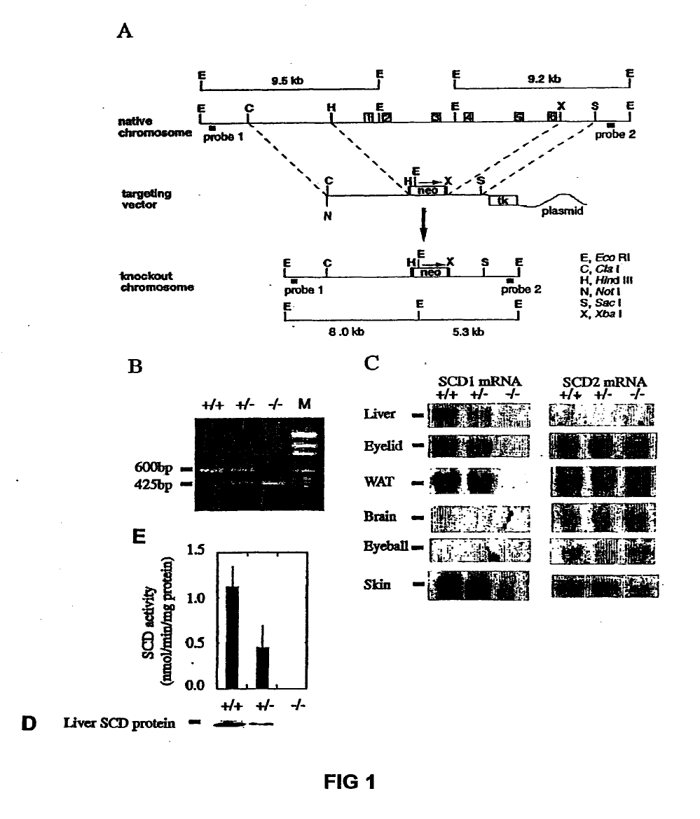

FIG. 1A shows the strategy used to knock out the SCD1 gene. The mouse SCD1 gene includes 6 exons. The first 6 exons of the gene were replaced by a neomycin-resistant cassette by homologous recombination, resulting in the replacement of the complete coding region of the SCD1 gene (FIG. 1A). The vector was electroporated into embryonic stem cells and the clones that integrated the neo cassette were selected by growth on geneticin. Targeted ES clones were injected into C57BV6 blastocysts yielding four lines of chimeric mice that transmitted the disrupted allele through the germ-line. The mutant mice were viable and fertile and bred with predicted Mendelian distributions. A PCR based screen to assay successful gene targeting of the SCD1 locus is shown in FIG. 1B. To determine whether the expression of the SCD1 gene was ablated we performed Northern blot analysis (FIG. 1C). SCD1 mRNA is undetectable in liver of SCD1−/− mice and reduced by approximately 50% in SCD+/− mice. SCD2 mRNA was expressed at low levels in both SCD1−/− mice and wild-type mice. Consistent with Northern blot results, Western blot analysis showed no immunoreactive SCD protein in liver from SCD−/− mice, whereas SCD1 protein was detectable in both heterozygous and wild-type liver tissue in a manner dependent on gene dosage. SCD enzyme activity in liver, as measured by the rate of conversion of [1-14C]stearoyl-CoA to [1-14C]oleate (FIG. 1E) was high in the wild-type mice but was undetectable in the total extracts of livers of the SCD1−/− mice.

Lipid Analysis

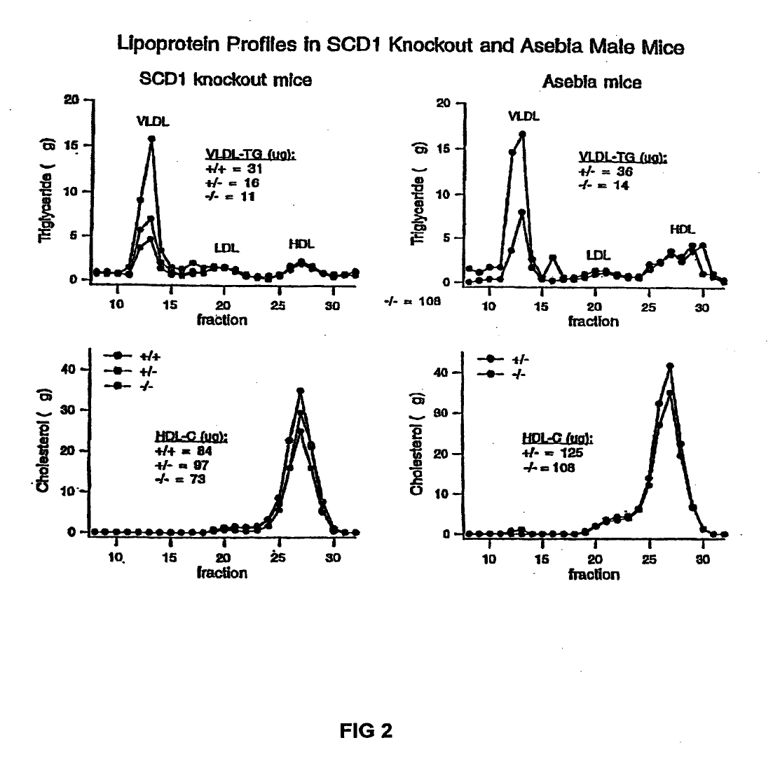

Analysis of liver cholesterol ester (0.8±0.1 vs. 0.3±0.1 mg/g liver) and liver triglycerides (12.6±0.3 vs. 7.5±0.6 mg/g liver) showed that SCD1 KO animals have lower amounts of both cholesterol esters and triglycerides than wild-type controls. Plasma lipoprotein analysis showed a decrease in plasma triglycerides (120.6±6.8 vs. 45.4±3.8) in SCD−/− mice compared to normal controls. These findings are similar to findings in Asebia mice. FIG. 2 records the plasma lipoprotein profile obtained using fast performance liquid chromatography. SCD1 Knock-Out mice showed a 65% reduction of triglyceride in VLDL fraction; but little or no significant difference in LDL or HDL levels.

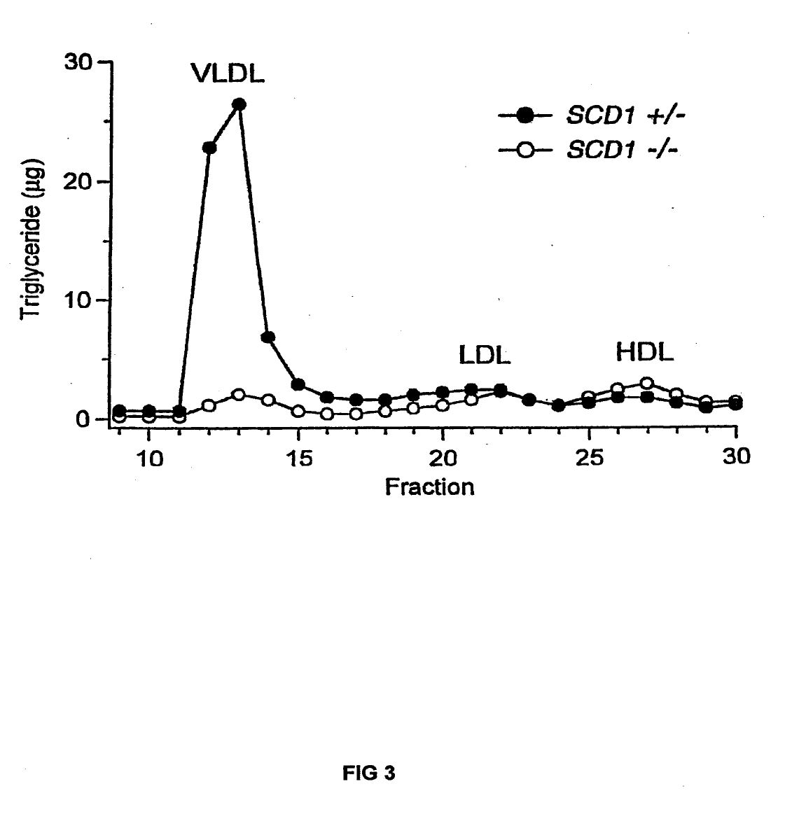

Asebia mice are compared with the SCD1 Knock-Out mice in FIG. 2. The findings are remarkably similar. Asebia mice plasma lipoproteins were separated by fast performance liquid chromatography and the distribution of triglycerides among lipoproteins in the various density fractions of the mice (n=3) are shown. FIG. 3 shows an additional example of an Asebia mouse lipoprotein profile. These profiles showed a major difference in the distribution of triglycerides in the VLDL fraction of the SCD−/− and SCD−/+ mice. The levels of triglycerides in the SCD−/+ were 25 mg/dl in the VLDL, with very low levels in the LDL and HDL fractions. In contrast the SCD−/− had very low levels of triglycerides in the three lipoprotein fractions.

Fatty Acid Analysis

We also determined the levels of monounsaturated fatty acids in various tissues. Table 1 shows the fatty acid composition of several tissues in wild-type and SCD−/− mice. The relative amounts of palmitoleate (16:1n-7) in liver and plasma from SCD−/− mice decreased by 55% and 47% while those of oleate (18:1n-9) decreased by 35% and 32%, respectively. The relative amount of palmitoleate in white adipose tissue and skin of SCD−/− mice were decreased by more than 70%, whereas the reduction of oleate in these tissues was less than 20% although the reduction was significant statistically. These changes in levels of monounsaturated fatty acids resulted in reduction of desaturation indices indicating reduction in desaturase activity. In contrast to these tissues, the brain, which expresses predominantly the SCD2 isoform, had a similar fatty acid composition and unaltered desaturation index in both wild type and SCD−/− mice. We conclude that SCD1 plays a major role in the production of monounsaturated fatty acids in the liver.

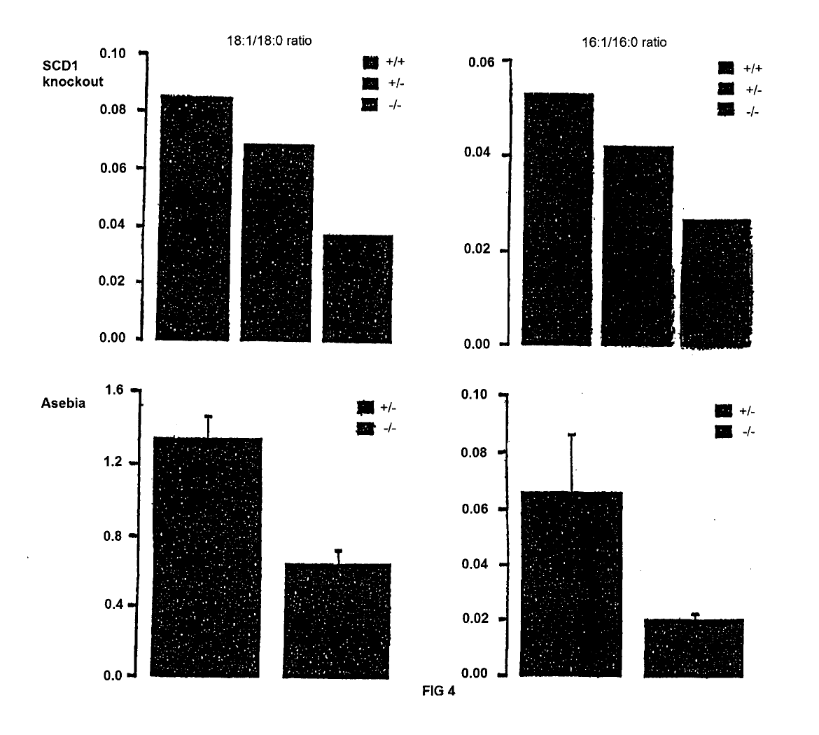

FIG. 4 (quantified in Table 2) demonstrate that SCD1 is a major contributor to the plasma desaturation indices (ratio of plasma 18:1/18:0 or 16:1/16:0 in the total lipid fraction), as judged by plasma fatty acid analysis of both the SCD1 KO and Asebia mice. In both animal models, a reduction of approximately 50% or greater is observed in the plasma desaturation indices. This demonstrates that the plasma desaturation index is highly dependent on the function of SCD1.

### Experimental Procedures for Knockout Mice:

Generation of the SCD1 Knockout Mice.

Mouse genomic DNA for the targeting vector was cloned from 129/SV genomic library. The targeting vector construct was generated by insertion of a 1.8-kb Xba I/Sac I fragment with 3′ homology as a short arm and 4.4-kb Cla I/Hind III fragment with 5′ homology cloned adjacent to neo expression cassette. The construct also contains a HSV thymidine kinase cassette 3′ to the 1.8-kb homology arm, allowing positive/negative selection. The targeting vector was linearized by Not I and electroporated into embryonic stem cells. Selection with geneticin and gancyclovior was performed. The clones resistant to both geneticin and gancyclovlor were analyzed by Southern blot after EcoRI restriction enzyme digestion and hybridized with a 0.4-kb probe located downstream of the vector sequences. For PCR genotyping, genomic DNA was amplified with primer A

| | |
|---|---|
| 5′-GGGTGAGCATGGTGCTCAGTCCCT-3′ | (SEQ ID NO: 2) |

which is located in exon 6, primer B

| | |
|---|---|
| 5′-ATAGCAGGCATGCTGGGGAT-3′ | (SEQ ID NO: 3) |

which is located in the neo gene (425 bp product, targeted allele), and primer C

| | |
|---|---|
| 5′-CACACCATATCTGTCCCCGACAAATGTC-3′ | (SEQ ID NO: 4) |

which is located in downstream of the targeting gene (600 bp product, wild-type allele). PCR conditions were 35 cycles, each of 45 sec at 94° C., 30 sec at 62° C., and 1 min at 72° C. The targeted cells were microinjected into C57BI/6 blastocysts, and chimeric mice were crossed with C57BU6 or 129/SvEv Taconic females, and they gave the germ-line transmission. Mice were maintained on a 12-h dark/light cycle and were fed a normal chow diet, a semi-purified diet or a diet containing 50% (% of total fatty acids) triolein, tripalmitolein or trieicosenoin. The semi-purified diet was purchased from Harlan Teklad (Madison, Wis.) and contained: 20% vitamin free casein, 5% soybean oil, 0.3% L-cystine, 13.2% Maltodextrin, 51.7% sucrose, 5% cellulose, 3.5% mineral mix (AIN-93G-MX), 1.0% vitamin mix (AIN-93-VX), 0.3% choline bitartrate. The fatty acid composition of the experimental diets was determined by gas liquid chromatography. The control diet contained 11% palmitic acid (16:0), 23% oleic acid (18:1n-9), 53% linoleic acid (18:2n-6) and 8% linolenic acid (18:3n-3). The high triolein diet contained 7% 16:0, 50% 18:1n-9, 35% 18:2n-6 and 5% 18:3n-3.

Materials

Radioactive [-³²P]dCTP (3000 Ci/mmol) was obtained from Dupont Corp. (Wilmington, Del.). Thin layer chromatography plates (TLC Silica Gel G60) were from Merck (Darmstadt, Germany). [1-14C]-stearoyl-CoA was purchased from American Radlolabeled Chemicals, Inc. (St Louis, Mo.) ImmoQilon-P transfer membranes were from Millipore (Danvers, Mass.). ECL Western blot detection kit was from Amersham-Pharmacia Biotech, Inc. (Piscataway, N.J.). All other chemicals were purchased from Sigma (St Louis, Mo.).

Lipid Analysis

Total lipids were extracted from liver and plasma according to the method of Bligh and Dyer (Bligh and Dyer, 1959), and phospholipids, wax esters, free cholesterol, triglycerides and cholesterol esters were separated by silica gel high performance TLC. Petroleum hexane/diethyl ether/acetic acid (80:30:1) or benzene/hexane (65:35) was used as a developing solvent (Nicolaides and Santos, 1985). Spots were visualized by 0.2% 2′,7′-dichlorofluorecein in 95% ethanol or by 10% cupric sulfate in 8% phosphoric acid. The wax triester, cholesterol ester and triglyceride spots were scraped, 1 ml of 5% HCI-methanol added and heated at 100° C. for 1 h (Miyazaki et al., 2000). The methyl esters were analyzed by gas-liquid chromatography using cholesterol heptadecanoate, triheptadecanoate and heptadecanoic acid as internal standard. Free cholesterol, cholesterol ester and triglycerides contents of eyelid and plasma were determined by enzymatic assays (Sigma St Louis, Mo. and Wako Chemicals, Japan).

Isolation and Analysis of RNA

Total RNA was isolated from livers using the acid guanidinium-phenol-chloroform extraction method (Bernlohr et al., 1985). Twenty micrograms of total RNA was separated by 1.0% agarose/2.2 M formaldehyde gel electrophoresis and transferred onto nylon membrane. The membrane was hybridized with 32P-labeled SCD1 and SCD2 probes. pAL 15 probe was used as control for equal loading (Miyazaki, M., Kim, Y. C., Gray-Keller, M. P., Attie, A. D., and Ntambi, J. M. (2000). The biosynthesis of hepatic cholesterol esters and triglycerides is impaired in mice with a disruption of the gene for stearoyl-CoA desaturase 1. J Biol Chem 275, 30132-8).

SCD Activity Assay

Stearoyl-CoA desaturase activity was measured in liver microsomes essentially as described by Shimomura et al. (Shimomura, I., Shimano, H., Kom, B. S., Bashmakov, Y., and Horton, J. D. (1998). Nuclear sterol regulatory element-binding proteins activate genes responsible for the entire program of unsaturated fatty acid biosynthesis in transgenic mouse liver. J Biol Chem 273, 35299-306.). Tissues were homogenized in 10 vol. of buffer A (0.1M potassium buffer, pH 7.4). The microsomal membrane fractions (100,000×g pellet) were isolated by sequential centrifugation. Reactions were performed at 37° C. for 5 min with 100 μg of protein homogenate and 60 μM of [1-14C]-stearoyl-CoA (60,000 dpm), 2 mM of NADH, 0.1M of Tris/HCl buffer (pH 7.2). After the reaction, fatty acids were extracted and then methylated with 10% acetic chloride/methanol. Saturated fatty acid and monounsaturated fatty acid methyl esters were separated by 10% AgNO3-impregnated TLC using hexane/diethyl ether (9:1) as developing solution. The plates were sprayed with 0.2% 2′,7′-dichlorofluorescein in 95% ethanol and the lipids were identified under UV light. The fractions were scraped off the plate, and the radioactivity was measured using a liquid scintillation counter. The enzyme activity was expressed as nmole min⁻¹ mg⁻¹ protein.

Immunoblotting

Pooled liver membranes from 3 mice of each group were prepared as described by Heinemann et al (Heinemann and Ozols, 1998). The same amount of protein (25 μg) from each fraction was subjected to 10% SDS-polyacrylamide gel electrophoresis and transferred to Immobilon-P transfer membranes at 4° C. After blocking with 10% non-fat milk in TBS buffer (pH 8.0) plus Tween at 4° C. overnight, the membrane was washed and incubated with rabbit anti-rat SCD as primary antibody and goat anti-rabbit IgG-HRP conjugate as the secondary antibody. Visualization of the SCD protein was performed with ECL western blot detection kit.

Histology

Tissues were fixed with neutral-buffered formalin, embedded in paraffin, sectioned and stained with hematoxylin and eosin.

Experimental Procedures for Asebia Mice:

Animals and Diets-Asebia homozygous (ab J/ab J or −/−) and heterozygous (+/ab J or +/−) mice were obtained from the Jackson Laboratory (Bar Harbor, Me.) and bred at the University of Wisconsin Animal Care Facility. In this study, comparisons are made between the homozygous (−/−) and the heterozygous (+/−) mice since the latter are indistinguishable from normal mice. Mice were housed in a pathogen-free barrier facility operating a 12-h light/12-h dark cycle. At 3 weeks of age, these mice were fed ad libitum for 2 wks or 2 months, on laboratory chow diet or on a semi-purified diet containing 50% (% of total fatty acids) triolein or tripalmitolein. The semi-purified diet was purchased from Harlan Teklad (Madison, Wis.) and contained: 18% vitamin free casein, 5% soybean oil, 33.55% com starch, 33.55% sucrose, 5% cellulose, 0.3% -L methionine, 0.1% choline chloride, salt mix (AIN-76A) and vitamin mix (AIN-76A). The fatty acid composition of the experimental diets was determined by gas liquid chromatography. The control diet contained 11% palmitic acid (16:0), 23% oleic acid (18:1n-9), 53% linoleic acid (18:2n-6) and 8% linoleic acid (18:3n-3). The high triolein diet contained 7% 16:0, 50% 18: In-9, 35% 18:2n-6 and 5% 18:3n-3. The high tripalmitolein diet contained 6% 16:0, 49% palmitoleic acid (16:1n-7), 12% 18:1n-9, 27% 18:2n-6 and 4% 18:3n-3.

Animals were anesthetized at about 10:00 a.m. by intraperitoneal injection of pentobarbital sodium (0.08 mg/g of body weight) Nembutal, Abbot, North Chicago, Ill.). Liver was isolated immediately, weighed, and kept in liquid nitrogen. Blood samples were obtained from the abdominal vein.

Materials-Radioactive α-32P]dCTP (3000 Ci/mmol) was obtained from Dupont Corp. (Wilmington, Del.). Thin layer chromatography plates (TLC Silica Gel G60) were from Merck (Darmstadt, Germany). [1-14C]-stearoyl-CoA, [3H]cholesterol and [1-14C]oleoyl-CoA were purchased from American Radiolabeled Chemicals, Inc. (St Louis, Mo.) Immobilon-P transfer membranes were from Millipore (Danvers, Mass.). ECL Western blot detection kit was from Amersham-Pharmacia Biotech, Inc. (Piscataway, N.J.). LT-1 transfection reagent was from PanVera (Madison, Wis.). All other chemicals were purchased from Sigma (St Louis, Mo.). The antibody for rat liver microsome SCD was provided by Dr. Juris Ozols at University of Connecticut Health Center. pcDNA3-1 expression vector SCD1 was provided by Dr. Trabis Knight at Iowa State University.

Lipid Analysis-Total lipids were extracted from liver and plasma according to the method of Bligh and Dyer (Bligh, E. G., and Dyer, W. J. (1959) Can J Biochem Physiol 37, 911-917.), and phospholipids, free cholesterol, triglycerides and cholesterol esters were separated by silica gel TLC. Petroleum ether/diethyl ether/acetic acid (80:30:1) was used as a developing solvent. Spots were visualized by 0.2% 2′,7′-dichlorofluorecein in 95% ethanol or by 10% cupric sulfate in 8% phosphoric acid. The phospholipid, cholesterol ester and triglyceride spots were scraped, 1 ml of 5% HCl-methanol added and heated at 100° C. for 1 h. The methyl esters were analyzed by gas-liquid chromatography using cholesterol heptadecanoate as internal standard (Lee, K. N., Pariza, M. W., and Ntambi, J. M. (1998) Biochem. Biophys. Res. Commun 248,817-821; Miyazaki, M., Huang, M. Z., Takemura, N., Watanabe, S., and Okuyama, H. (1998) Lipids 33, 655-61). Free cholesterol, cholesterol ester and triglycerides contents of liver and plasma were determined by enzymatic assays (Sigma St Louis, Mo. and Wako Chemicals, Japan).

Plasma Lipoprotein Analysis-Mice were fasted a minimum of 4 hours and sacrificed by CO₂ asphyxiation and/or cervical dislocation. Blood was collected aseptically by direct cardiac puncture and centrifuged (13,000×g, 5 min, 4° C.) to collect plasma. Lipoproteins were fractionated on a Superose 6HR 10/30 FPLC column (Pharmacla). Plasma samples were diluted 1:1 with pes, filtered (Cameo 3AS syringe filter, 0.22 um) and injected onto the column that had been equilibrated with PBS containing 1 mM EDTA and 0.02% NaN₃. The equivalent of 100 μl of plasma was injected onto the column. The flow rate was set constant at 0.3ml/min. 500 μl fractions were collected and used for total triglyceride measurements (Sigma). Values reported are for total triglyceride mass per fraction. The identities of the lipoproteins have been confirmed by utilizing anti-ApoB immunoreactivity for LDL and Anti-Apo A1 immnunoreactivity for HDL (not shown).

### EXAMPLE 2

### Lower Body Fat Level and Increased Lean Body Mass in SCD1 Knock-Out Mice

In this example, SCD1 knock-out mice (SCD1−/−) generated as described in Example 1 and their age-, weight- and sex-matched wild-type counterparts (SCD1+/+) were fed with either a regular chow diet or a high fat diet for 23 weeks. At the end of the 23-week, body weights and body fat levels were compared between the SCD1−/− mice and the SCD1+/+ mice. Body weights and body fat levels were also compared between SCD1−/− mice or SCD1+/+ mice that were on the regular chow diet or high fat diet.

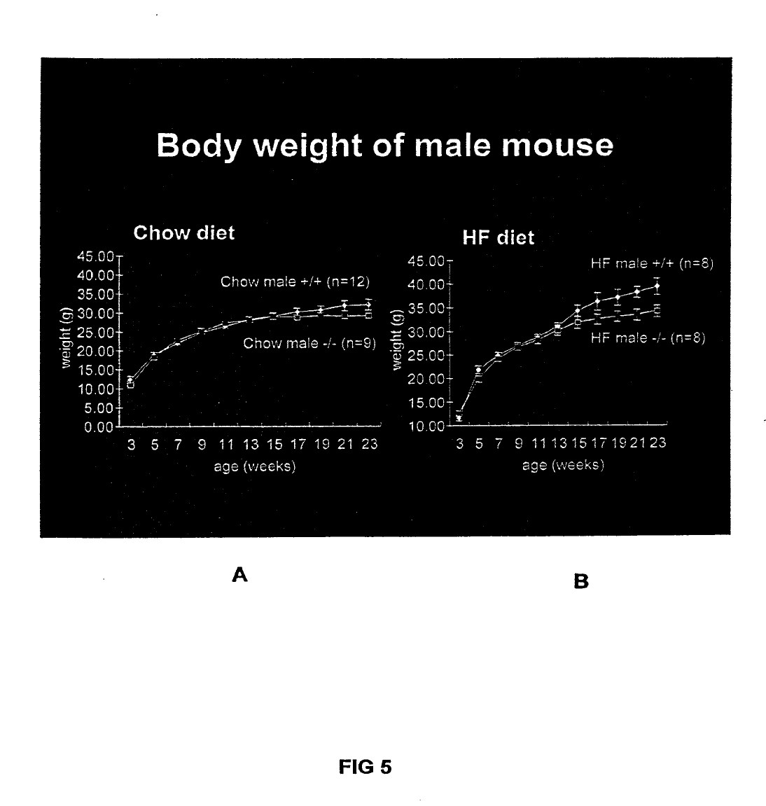

As shown in FIG. 5A, SCD1−/− male mice and SCD1+/+ male mice fed the regular chow diet had similar average body weight. SCD1+/+ mice fed the high fat diet had higher average body weight than SCD1−/− mice on the same diet (p<0.03) (FIG. 5B). However, SCD1−/− mice fed the high fat diet had comparable average body weight to SCD1−/− mice fed the regular chow diet indicating that SCD1−/− mice were resistant to diet-induced obesity.

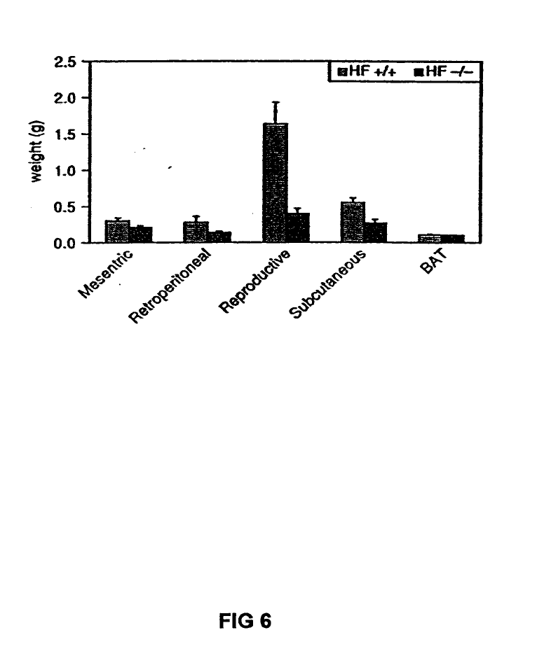

The epidydimal fat pad size was also dramatically reduced in the SCD1−/− mice fed the regular chow diet in comparison to SCD1+/+ mice fed the same diet. Further, on the regular chow diet, the weights of various fat pads in the SCD1−/− mice were lower than the SCD1+/+ mice (FIG. 6). On the high fat diet, the weight difference between various corresponding fat pads in SCD1−/− and SCD1+/+ mice became even bigger.

When carcass protein content was measured in SCD1−/− and SCD1+/+ mice, it was found that carcass protein constituted 18% and 16.8% of body weight for SCD1+/+ and SCD1−/− mice, respectively (p<0.05).

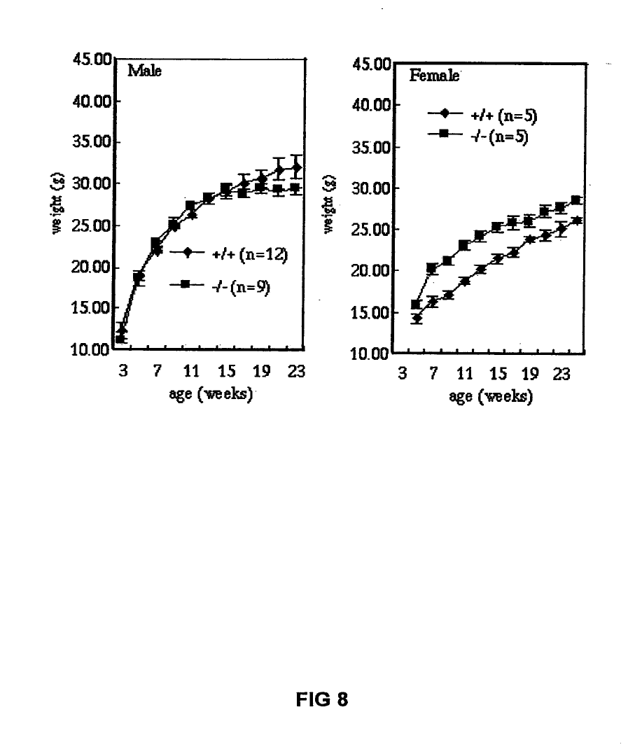

Shown in FIG. 8 are results from another trial. In this trial, the female SCD1 deficient knockout mice were significantly heavier and had more body mass than the comparable controls, when both experimentals and controls were fed the same diet. This data, when taken in conjunction with the data demonstrating that lean body mass increases in the SCD1 activity deficient mice, suggests a significant increase in lean body mass in these female mice.

### EXAMPLE 3

### Transcriptional Regulators of SCD1 and Their Use as Drug Screening Targets

This example reports the complete genomic promoter sequence of human SCD1. This promoter is used herein to identify regulatory elements that modulate and control SCD1 expression in humans, and identifies regulatory proteins that are suitable targets for small molecule intervention to modulate expression of SCD1 in humans.

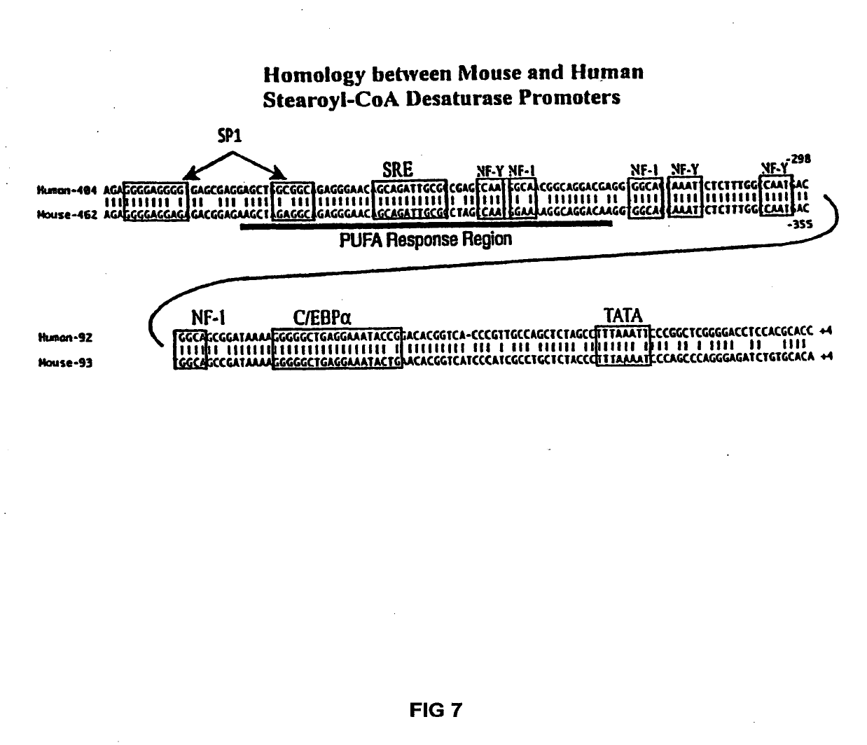

The human SCD1 promoter sequence is set forth at SEQ ID No. 1. FIG. 7 illustrates the location of regulatory sequences and binding sites in the homologous region of the mouse SCD1 and human SCD1 promoter and 5′-flanking regions. The top scale denotes the position relative to the transcriptional start site. Important promoter sequence elements are indicated.

The human SCD1 promoter structure is similar to that of the mouse SCD1 isoform and contains conserved regulatory sequences for the binding of several transcription factors, including the sterol regulatory element binding protein (SREBP), CCAA T enhancer binding protein-alpha (C/EBPa) and nuclear factor-1 (NF-1) that have been shown to transactivate the transcription of the mouse SCD gene. Cholesterol and polyunsaturated fatty acids (PUFAs) decreased the SCD promoter-luciferase activity when transiently transfected into HepG2 cells. The decrease in promoter activity in the reporter construct correlated with decreases in endogenous SCD mRNA and protein levels. Transient co-transfection into HepG2 cells of the human SCD promoter-luciferase gene construct together with expression vector for SREBP revealed that SREBP trans-activates the human SCD promoter. Our studies indicate that like the mouse SCD1 gene, the human SCD gene is regulated by polyunsaturated fatty acids and cholesterol at the level of gene transcription and that SREBP plays a role in the transcriptional activation of this gene.

Construction of the Chimeric Promoter Luciferase Plasmid

A human placenta genomic library in bacteria-phage I EMBL3 was screened with a 2.0 kb Pstl insert of the mouse pC3 cDNA (Ntambi, J. M., Buhrow, S. A., Kaestner, K. H., Christy, R. J., Sibley, E., Kelly, T. J. Jr., and M. D. Lane. 1988. Differentiation-induced gene expression in 3T3-L1 preadipocytes: Characterization of a differentially expressed gene encoding stearoyl-CoA desaturase. J. Biol. Chem. 263: 17291-17300.) as a radioactive probe and seven plaques were isolated. Two of these plaques were purified to homogeneity, the DNA isolated and designated HSCD1 and HSCD3. A DNA primer based on the sequence corresponding to the first exon of the cDNA of the published human stearoyl-CoA desaturase gene (Zhang, L., G. E. Lan, S. Parimoo, K. Stenn and S. M. Proutey. 1999. Human stearoyl-CoA desaturase: alternative transcripts generated from a single gene by usage of tandem polyadenylation sites. Biochem. J. 340: 255-264) was synthesized and used to sequence the two phage clones by the dideoxy nucleotide chain termination method. A preliminary sequence was generated and primers upstream:

SEQ ID No. 5
5′ NNNNGGTACCTTNNGAAAAGAACAGCGCCC 3′

and downstream:

| | |
|---|---|
| 5′ NNNNAGATCTGTGCGTGGAGGTCCCCG 3′ | SEQ ID No. 6 |

were designed to amplify approximately 540 bases of the promoter region upstream of the transcription start site: These primers contain inserted restriction enzyme sites (underlined), Kpn1 for upstream, and BgIII for downstream, with a 4 base overhang region to allow restriction enzyme digestion. PCR was then performed on the phage clones and the amplified 500 bp fragment was isolated from a 1% agarose gel.

The amplified fragment was digested with Kpn1 and BgIII and then cloned into the Kpn1 and BgIII sites of the pGL3 basic vector (Promega) that contains the luciferase reporter gene and transformed into DH5 competent E. coli cells. Plasmid DNA was purified on Qiagen columns and sequenced by the dideoxynucleotide chain termination method using as primers corresponding to DNA sequences within the multiple cloning site but flanking the inserted DNA. The SCD promoter luciferase gene construct that was generated was designated as pSCD-500.

Isolation and Analysis of RNA-Total RNA was isolated from HepG2 cells using the acid guanidinium-phenol-chloroform extraction method. Twenty micrograms of total RNA was separated by 0.8% agarose/2.2 M formaldehyde gel electrophoresis and transferred onto nylon membrane. The membrane was hybridized with 32P-labeled human SCD cDNA probe generated by PCR as follows: pAL 15 probe was used as control for equal loading.

Immunoblotting—Cell extracts were prepared from HepG2 cells that had been treated with the various fatty acids or cholesterol as described by Heinemann et al (17). The same amount of protein (60 μg) from each fraction was subjected to 10% SDS-polyacrylamide gel electrophoresis and transferred to Immobilon-P transfer membranes at 4° C. After blocking with 10% non-fat milk in TBS buffer (pH 8.0) plus 0.5% Tween at 4° C. overnight, the membrane was washed and incubated with rabbit anti-rat SCD as primary antibody (17) and goat anti-rabbit IgG-HRP conjugate as the secondary antibody. Visualization of the SCD protein was performed with ECL western blot detection kit.

Effect of Cholesterol, Polyunsaturated Fatty Acids and Arachidonic Acidon the Expression of hSCD1

Cell Culture and DNA transfections—HepG2 cells, were grown in Low Glucose DMEM supplemented with 10% Fetal Bovine Serum and 1% Penicillin/Streptomycin solution and maintained at 37° C., 5% CO2 in a humidified incubator. Cells were passaged into 6 cm dishes to give 40-70% confluence in about 12-16 hours. Cells were then transfected with 5 μg plasmid DNA per plate of pSCD-500 or the Basic PGL3 reporter as well as well as the pRL-TK, internal controls (Promega) using the LT-1 transfection reagent (Pan Vera). After 48 hours, cells were rinsed with PBS and then treated in Williams' E Media, a fatty acid-free media, containing insulin, dexamethasone, and appropriate concentrations of albumin-conjugated fatty acids as indicated in figures and legends. Cells were also treated with ethanol alone (as control) or cholesterol (10 μg/mL) and 25-OH cholesterol (1 μg/mL) dissolved in ethanol. After an additional 24 h, extracts were prepared and assayed for luciferase activity. Non-transfected cells were used as the blank and Renilla Luciferase was used as an internal control. Cell extracts were assayed for protein according to Lowry, and all results were normalized to protein concentration as well as to renilla luciferase counts. Each experiment was repeated at least three times, and all data are expressed as means±SEM.

When compared to the mouse SCD1 promoter sequence, it was found that several functional regulatory sequences identified in the mouse SCD1 promoter are absolutely conserved at the nucleotide level and also with respect to their spacing within the proximal promoters of the two genes (FIG. 7). Both the TTAATA homology, the C/EBPa and NF-1 are in the same locations in both the mouse SCD1 and human promoters. Further upstream the sterol regulatory element (SRE) and the two CCAAT box motifs that are found in the polyunsaturated fatty acid responsive element (PUFA-RE) of the mouse SCD1 and SCD2 promoters. The spacing of these elements is conserved in the three promoters.

We tested whether the human SCD gene expression was also repressed by cholesterol and polyunsaturated fatty acids. Human HepG2 cells were cultured and then treated with 100 μM arachidonic acid, DHA or 10 μg/ml cholesterol and 1 μg/ml of 25-hydroxycholesterol cholesterol as we have described previously. Total mRNA was isolated and subjected to northern blot analysis using a probe corresponding to the human cDNA and generated by the PCR method using primers based on published human SCD cDNA sequence. It has been shown that AA, DHA and cholesterol decreased the human SCD mRNA expression in a dose dependent manner. The western blot of the protein extracts of the cells treated with PUFAs and cholesterol shows that PUFAs and cholesterol decreased the levels of the SCD protein as well (data not shown).

To assess the possible effect of SREBP binding on the activity of the human SCD promoter the human luciferase promoter construct was co-transfected in HepG2 cells together with an expression vector containing SREBP1a. After 72 h, extracts of the transfected cells were assayed for luciferase activity. Data were normalized to cell extract expressing the Renilla luciferase as an internal control. SREBP transactivates the promoter in a dose dependent manner giving rise to an increase up to 40-fold. This experiment shows that SREBP plays a role in regulating the human SCD gene.

Published reports indicated that the mature form of SREBP, in addition to activating the lipogenic genes, also mediates PUFA and cholesterol repression of lipogenic genes, including mouse SCD1. To observe the regulatory effects of mature SREBP-1a and PUFAs on the activity of SCD promoters, HepG2 hepatic cells were transiently co-transfected with 20 ng (per 6-cm dish) of plasmid DNA containing the human SCD promoter as described above but this time the transfectlons were carried out in the presence of cholesterol to inhibit the maturation of the endogenous SREBP and thus ensure that there was little mature form of the endogenous SREBP present in the cells. After transfection, the cells were then treated with, arachidonic acid, EPA and DHA as albumin complexes and luciferase activity was then assayed using a luminometer. If SREBP mediates PUFA repression of the human SCD gene, SCD promoter activity would not diminish upon treatment the transfected cells with PUFA. However addition of AA, EPA or DHA continued to repress SCD promoter activity with only a slight attenuation (data not shown). Thus, SREBP maturation does not seem to exhibit the selectivity required to explain PUFA control of SCD gene transcription suggesting that PUFA may utilize a different protein in addition to the SREBP to repress human SCD gene transcription.

These results establish that hSCD1 is transcriptionally regulated by SREBP, NF-Y, C/EBPalpha, PUFA-RE and alternate proteins and transcription regulators. Each one of these proteins is therefore be an attractive drug screening target for identifying compounds which modulate SCD1 expression in a cell; and thereby being useful for treating the human diseases, disorders and conditions which are taught by the instant invention.

The promoter sequence of human stearoyl-CoA desaturase 1 is set forth in published PCT applications WO01/62954, the disclosure of which is hereby incorporated by reference.

### REFERENCE LIST

1. Mihara, K. Structure and regulation of rat liver microsomal stearoyl-CoA desaturase gene. J. Biochem. (Tokyo) 108, 1022-1029 (1990).
2. Thiede, M. A. & Strittmatter,P. The induction and characterization of rat liver “stearyl-CoA desaturase mRNA. J. Biol. Chem. 260, 14459-14463 (1985).
3. Kaestner, K. H., Ntambi, J. M., Kelly, T. J., Jr. & Lane, M. D. Differentiation-Induced gene expression in 3T3-L 1 preadipocytes. A second differentially expressed gene encoding stearoyl-CoA desaturase. J. Biol. Chem. 264, 14755-14761 (1989).
4. Ntambi, J. M. et al. Differentiation-induced gene expression in 3T3-L 1 preadipocytes. Characterization of a differentially expressed gene encoding stearoyl- CoA desaturase. J. Biol. Chem. 263, 17291-17300 (1988).
5. Zhang, L., Ge, L., Parimoo, S., Stenn, K. & Prouty, S. M. Human stearoyl-CoA desaturase: alternative transcripts generated from a single gene by usage of tandem polyadenylation sites. Biochem. J. 340 (pt 1), 255-264 (1999).
6. Zheng, Y. et al. Scd1 is expressed in sebaceous glands and is disrupted in the asebia mouse [letter]. Nat. Genet. 23, 268-270 (1999).
7. Sundberg, J. P. et al. Asebia-2J (Scd1(ab2J)): a new allele and a model for scarring alopecia. Am. J. Pathol. 156, 2067-2075 (2000).
8. Miyazaki, M., Kim, Y. C., Keller, M. P., Attie, A. D. & Ntambi, J. M. The biosynthesis of hepatic cholesterol esters and trlglycerides is impaired in mice with a disruption of the gene for stearoyl-CoA desaturase 1. J. Biol. Chem. (2000).
9. Spector, A. A. & Yorek, M. A. Membrane lipid composition and cellular function. J. Lipid Res. 26,1015-1035 (1985).
10. Enser, M. & Roberts, J. L. The regulation of hepatic stearoyl-coenzyme A desaturase in obese-hyperglycaemic (ob/ob) mice by food intake and the fatty acid composition of the diet. Biochem. J. 206, 561-570 (1982).
11. Enser, M. The role of insulin in the regulation of stearic acid desaturase activity In liver and adipose tissue from obese-hyperglycaemic (ob/ob) and lean mice. Biochem. J. 180, 551-558 (1979).
12. Enser, M. Desaturation of stearic acid by liver and adipose tissue from obese-hyperglycaemic mice (ob/ob). Biochem. J. 148,551-555 (1975).
13. Jones, B. H. et al. Adipose tissue stearoyl-CoA desaturase mRNA is increased by obesity and decreased by polyunsaturated fatty acids. Am. J. Physiol 271, E44-E49 (1996).
14. Kim, Y. C., Gomez, F. E., Fox, B. G. & Ntambi, J. M. Differential regulation of the stearoyl-CoA desaturase genes by thiazolidinediones in 3T3-L1 adipocytes. J. Lipid Res. 41, 1310-1316 (2000).
15. Li, J. et al. Partial characterization of a cDNA for human stearoyl-CoA desaturase and changes in its mRNA expression in some normal and malignant tissues. Int. J. Cancer 57, 348-352 (1994).
16. Wood, C. B. et al. Reduction in the stearic to oleic acid ratio in human malignant liver neoplasms. Eur. J. Surg. Oncol. 11,347-348 (1985).
17. Habib, N. A. et al. Stearic acid and carcinogenesis. Br. J. Cancer 56, 455-458 (1987).
18. Tronstad, K. J., Berge, K., Bjerkvig, R., Flatmark, T. & Berge, R. K. Metabolic effects of 3-thia fatty acid in cancer cells. Adv. Exp. Med. Biol. 466, 201-204 (1999).
19. DeWille, J. W. & Farmer, S. J. Postnatal dietary fat influences mRNAS involved in myelination. Dev. Neurosci. 14,61-68 (1992).
20. Garbay, B. et al. Regulation of oleoyl-CoA synthesis in the peripheral nervous system: demonstration of a link with myelin synthesis. J. Neurochem. 71, 1719-1726 (1998).
21. Marcelo, C. L., Duell, E. A., Rhodes, L. M. & Dunham, W. R. In vitro model of essential fatty acid deficiency. J. Invest Dermatol. 99, 703-708 (1992).
22. Tebbey, P. W. & Buttke, T. M. Stearoyl-CoA desaturase gene expression in lymphocytes [published erratum appears in Biochem Biophys Res Commun 1992 Sep. 16; 187(2):1201]. Biochem. Biophys. Res. Commun 186,531-536 (1992).
23. Tebbey, P. W. & Buttke, T. M. Molecular basis for the immunosuppressive action of stearic acid on T cells [published erratum appears in Immunology 1990 October; 71(2):306] Immunology 70,379-386 (1990).
24. Stampfer et al. A prospective study of cholesterol, apolipoproteins, and the risk of myocardial infraction. N. Engl. J. Med. 325,373-381 (1991).
25. Schmidt et al. Clustering of dyslipidemia, hyperuricemia, diabetes, and hypertension and its association with fasting insulin and central and overall obesity in a general population. Atherosclerosis Risk in Communities Study Investigators Metabolism 45 (6):699-706 (1996).
26. Park et al Inhibition of hepatic stearoyl-CoA desaturase activity by trans-10, cls-12 conjugated linoleic acid and its derivatives. Biochim Biophys Acta. 1486(2-3):285-92 (2000).
27. Choi et al. The trans-10, cis-12 isomer of conjugated linoleic acid downregulates stearoyl-CoA desaturase 1 gene expression in 3T3-L1 adipocytes. J Nutr. 130(8):1920-4 (2000)

1.-18. (canceled)

19. A method for controlling body fat in a human or non-human subject, the method comprising the steps of:
administering to the subject an antisense oligonucleotide to stearoyl-CoA desaturase 1 (SCD1) messenger RNA sequence in an amount sufficient to control body fat in the subject.

20. The method of claim 19, wherein the antisense oligonucleotide is a DNA oligonucleotide.

21. The method of claim 19, wherein the antisense oligonucleotide is an RNA oligonucleotide.

22. The method of claim 19, wherein the antisense oligonucleotide is between about 20-25 nucleotides.

23. The method of claim 19, wherein the antisense oligonucleotide is directed against the 5′ end of an SCD1 messenger RNA.

24. The method of claim 19, wherein the antisense oligonucleotide comprises phosphorothioate derivatives.

25. The method of claim 19, wherein the antisense oligonucleotide is administered to the subject in a carrier.

26. The method of claim 25, wherein the carrier is a cationic liposome.

27. The method of claim 19, wherein the antisense oligonucleotide is expressed from a vector engineered to produce the antisense oligonucleotide.


SEQUENCE LISTING (6 sequences)

SEQ ID NO: 1 - DNA, 617 bp, Homo sapiens
ggtccccgccccttccagagagaaagctcccgacgcgggatgccgggcagaggcccagcggcgggtggaagagaagctgagaaggagaaacagaggggagggggagcgaggagctggcggcagagggaacagcagattgcgccgagccaatggcaacggcaggacgaggtggcaccaaattcccttcggccaatgacgagccggagtttacagaagcctcattagcatttccccagaggcaggggcaggggcagaggccgggtggtgtggtgtcggtgtcggcagcatccccggcgccctgctgcggtcgccgcgagcctcggcctctgtctcctccccctcccgcccttacctccacgcgggaccgcccgcgccagtcaactcctcgcactttgcccctgcttggcagcggataaaagggggctgaggaaataccggacacggtcacccgttgccagctctagcctttaaattcccggctcggggacctccacgcaccgcggctagcgccgacaaccagctagcgtgcaaggcgccgcggctcagcgcgtaccggcgggcttcgaaaccgcagtcctccggcgaccccgaactccgctccggagcctcagccccct

SEQ ID NO: 2 - DNA, 24 bp, Artificial Sequence
  Description of Artificial Sequence PCR Primer for Exon 6.
gggtgagcatggtgctcagtccct

SEQ ID NO: 3 - DNA, 20 bp, Artificial Sequence
  Description of Artificial Sequence PCR Primer for the neo gene.
atagcaggcatgctggggat

SEQ ID NO: 4 - DNA, 28 bp, Artificial Sequence
  Description of Artificial Sequence PCR amplification primer.
cacaccatatctgtccccgacaaatgtc

SEQ ID NO: 5 - DNA, 30 bp, Artificial Sequence
  Description of Artificial Sequence Upstream PCR amplification primer.
nnnnggtaccttnngaaaagaacagcgccc

SEQ ID NO: 6 - DNA, 27 bp, Artificial Sequence
  Description of Artificial Sequence Downstream PCR amplification primer.
nnnnagatctgtgcgtggaggtccccg